(19) United States
(12) Patent Application Publication
(10) Pub. No.: US 2012/0045749 A1
(43) Pub. Date: Feb. 23, 2012

(54) METHODS OF CATEGORIZING AN ORGANISM

(75) Inventor: Colin William Dykes, Albuquerque, NM (US)

(73) Assignee: OPGEN, INC., Gaithersburg, MD (US)

(21) Appl. No.: 13/147,056

(22) PCT Filed: Jan. 29, 2010
(86) PCT No.: PCT/US10/22574

§ 371 Date: Nov. 1, 2011

Related U.S. Application Data
(60) Provisional application No. 61/148,376, filed on Jan. 29, 2009.

(51) Int. Cl. C12Q 1/68; C12Q 1/70

(52) U.S. Cl. 435/5; 435/6.11

(57) ABSTRACT

The invention generally relates to methods of identifying and categorizing organisms and more specifically methods of generating and using patterns of chromosomal variation in order to classify organisms.

### RELATED APPLICATION

The present invention is related to and claims the benefit of U.S. provisional patent application Ser. No. 61/148,376, filed Jan. 29, 2009, the contents of which are incorporated by reference herein in their entirety.

### TECHNICAL FIELD

The invention generally relates to methods of identifying and categorizing organisms and more specifically methods of generating and using patterns of chromosomal variation in order to classify organisms.

### BACKGROUND

Rapid identification of microorganisms, such as bacteria, from clinical samples is important in clinical microbiology. Moreover, the proper classification and/or characterization of microorganisms can have a significant impact on proper diagnosis and treatment of disease.

Traditional methods for phylogenetic analysis of microorganisms at the DNA level involve creating restriction digests and using pulse-field electrophoresis to produce banding patterns that are useful in determining the relatedness of different microorganisms or different strains of a microorganism. Recently, optical mapping has enabled the generation of genomic restriction maps of many thousands of single DNA molecules. Each optical molecule map contains an ordered set of DNA fragments of distinct sizes. The order and sizes of the fragments within a given map represents a unique signature of the genome of the organism from which the DNA was obtained. optical mapping allows the collection of thousands of single molecule maps in parallel. optical mapping also has the benefit of allowing the identification of bacteria directly from clinical samples without the need for growth on primary culture medium.

The optical mapping technique has the benefit of conveying more information that standard electrophoresis, which only is able to separate fragments by size and charge. For example, optical mapping has the capability of differentiating characteristics of samples other than simply size. The present invention provides various novel uses for optical mapping in the identification and analysis of organisms.

### SUMMARY

The present invention provides methods of identifying and classifying organisms. Methods of the invention utilize optical mapping in order to provide insight into genomic characteristics of a microorganism, resulting in rapid identification and classification.

In one embodiment, methods of the invention allow the determination of the genetic relatedness of two or more organisms based upon optical maps produced from restriction digests of their DNA. The invention is particularly useful for the identification and classification of microorganisms, particularly disease-causing microorganisms. For example, methods of the invention have been used to identify patterns of chromosomal markers in antibiotic-resistant bacteria that allow classification of the bacteria with respect to specific resistance characteristics. That type of classification is useful for determining the appropriate course of treatment for an individual infected with the bacterium from which the DNA was obtained.

Methods of the invention also allow one to determine a likely lineage for a particular genomic element in a microorganism under investigation. For example, methods of the invention are useful for identifying the source of the antibiotic resistance in an isolated microorganism. Moreover, optical mapping according to the invention allows the identification of common genetic elements or patterns in organisms, such as microorganisms, that are informative with respect to the choice of treatment options. In the area of antibiotic resistance, one can also determine whether resistance was acquired, for example, by transfer via a conjugative plasmid or some other event or series of events.

Methods of the invention allow the identification of genomic rearrangements, such as inversions, that would not be observable using traditional techniques, such as pulse-gel electrophoresis. The ability to identify genomic changes at a level of granularity not before achieved opens up many new research and clinical applications, including establishing phylogenetic relationships, suggesting appropriate treatments, determining the etiology of disease, determining the way in which genomic elements (e.g., antibiotic resistance) are acquired and passed on, among others.

The invention contemplates, in one embodiment, creating patterns that are useful as markers of genomic characteristics of an organism. Pattern generation and comparison is a useful way to categorize microorganisms, such as bacteria, and to create catalogs of strains or types based upon relevant genetic characteristics. For example, bacteria can be classified on the basis of patterns generated by optical mapping with respect to their antibiotic resistance properties. Generating the patterns and then comparing unknown samples leads to rapid and accurate diagnosis followed by appropriate treatment. Using methods of the invention, one can determine whether a specific bacterium is vancomycin-resistance, methicillin-resistant and, if so, what subtype (e.g., hospital-acquired vs. community acquired).

In another embodiment, the invention contemplates obtaining DNA from an organism (e.g., a test organism), creating restriction fragments of the DNA and making an optical map based upon those fragments. The optical map is then compared to maps of restriction fragments of at least on other organism in order to categorize the test organism. By categorization, it is meant placing the organism in a category based upon patterns in the optical map. Categorization can be done by similarities or differences in one or more pattern(s) present in the optical map of the organism and those of organisms in a database or other organisms for which optical maps are created in concert with the test organism.

The invention allows the determination of the relatedness of organisms, such as microorganisms based upon the pattern of restriction fragments, or markers, on nucleic acid obtained from the organism(s). FIG. 9, for example, shows the pattern of deletions, insertions, inversions and repeats in nine strains of vancomycin-resistant stapholoccus aureus (VRSA). The various triangles in the schematic indicate spots in which a deletion or insertion has occurred. These were determined to be characteristic of the particular strain that displayed resistance. These patterns allow one to determine that VRSA 1 and VRSA 5 are the same. More importantly, the patterns across all nine strains reveal that the vancomycin resistant trait did not originate from the same progenitor source. This conclusion has importance in tracing the source of an infection and in matching the treatment with the particular bacterium. It is important to note that it is immaterial for purposed of the invention exactly what the deletion or insertion is (i.e., what particular nucleotides were deleted or inserted). Rather, what is important is the pattern of insertions and/or deletions along the length of the chromosome. It is those patterns that allow one to compare strains, subtypes, etc. in order to make determinations about phylogeny, categorization, etiology, and the like.

Methods of the invention are based upon chromosomal DNA analysis using optical mapping, which produces high-resolution, ordered restriction maps of an organisms genome. Once prepare, as detailed below, maps are compared, for example, by using phylogenetic analysis techniques and viewers as described herein. Patterns produced using optical maps of the invention are useful to distinguish, categorize, and compare the organisms from which DNA was obtained.

In one aspect, an unknown sample is compared to a database of optical maps, or patterns generated therefrom, in order to allow identification, classification, comparison, etc. of organisms. Using a restriction map database, organisms are identified and classified not just at a genus and species level, but also at a sub-species (strain), a sub-strain, and/or an isolate level. The featured methods offer fast, accurate, and detailed information for identifying and classifying organisms. Methods of the invention can be used in a clinical setting, e.g., a human or veterinary setting; or in an environmental or industrial setting (e.g., clinical or industrial microbiology, food safety testing, ground water testing, air testing, contamination testing, and the like). In essence, the invention is useful in any setting in which the detection and/or identification of a microorganism is necessary or desirable.

This invention also features methods of diagnosing a disease or disorder in a subject by, inter alia, identifying at least one organism by correlating the restriction map of a nucleic acid from each organism with a restriction map database and correlating the identity of each organism with the disease or disorder. Methods of the invention further contemplate using the diagnosis to prescribe appropriate treatment.

The DNA from any organism can be used in methods of the invention. Common organism include a microorganism, a bacterium, a protist, a virus, a fungus, or disease-causing organisms including microorganisms such as protozoa and multicellular parasites. The nucleic acid can be deoxyribonucleic acid (DNA), a ribonucleic acid (RNA) or can be a cDNA copy of an RNA obtained from a sample. The nucleic acid sample includes any tissue or body fluid sample, environmental sample (e.g., water, air, dirt, rock, etc.), and all samples prepared therefrom.

Methods of the invention can further include digesting nucleic acid with one or more enzymes, e.g., restriction endonucleases, e.g., BglII, NcoI, XbaI, and BamHI, prior to imaging. Preferred restriction enzymes include, but are not limited to:

| | | |
|---|---|---|
| AflII | ApaLI | BglII |
| AflII | BglII | NcoI |
| ApaLI | BglII | NdeI |
| AflII | BglII | MluI |
| AflII | BglII | PacI |
| AflII | MluI | NdeI |
| BglII | NcoI | NdeI |
| AflII | ApaLI | MluI |
| ApaLI | BglII | NcoI |
| AflII | ApaLI | BamHI |
| BglII | EcoRI | NcoI |
| BglII | NdeI | PacI |
| BglII | Bsu36I | NcoI |
| ApaLI | BglII | XbaI |
| ApaLI | MluI | NdeI |
| ApaLI | BamHI | NdeI |
| BglII | NcoI | XbaI |
| BglII | MluI | NcoI |
| BglII | NcoI | PacI |
| MluI | NcoI | NdeI |
| BamHI | NcoI | NdeI |
| BglII | PacI | XbaI |
| MluI | NdeI | PacI |
| Bsu36I | MluI | NcoI |
| ApaLI | BglII | NheI |
| BamHI | NdeI | PacI |
| BamHI | Bsu36I | NcoI |
| BglII | NcoI | PvuII |
| BglII | NcoI | NheI |
| BglII | NheI | PacI |

Imaging ideally includes labeling the nucleic acid. Labeling methods are known in the art and can include any known label. However, preferred labels are optically-detectable labels, such as 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron® Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; naphthalo cyanine, BOBO, POPO, YOYO, TOTO and JOJO.

A database for use in the invention can include a restriction map similarity cluster. The database can include a restriction map from at least one member of the clade of the organism. The database can include a restriction map from at least one subspecies of the organism. The database can include a restriction map from a genus, a species, a strain, a sub-strain, or an isolate of the organism. The database can include a restriction map with motifs common to a genus, a species, a strain, a sub-strain, or an isolate of the organism.

In another aspect, the invention features a method of diagnosing a disease or disorder in a subject, including obtaining a sample suspected to contain at least one organism to be detected; (b) imaging a nucleic acid from each organism; (c) obtaining a restriction map of each nucleic acid; (d) identifying each organism by correlating the restriction map of each nucleic acid with a restriction map database; and (e) correlating the identity of each organism with the disease or disorder or with other organisms in the database.

Methods can further include treating a disease or disorder in a subject, including diagnosing a disease or disorder in the subject as described above and providing treatment to the subject to ameliorate the disease or disorder. Treatment can include administering a drug to the subject.

In one embodiment, a restriction map obtained from a single DNA molecule is compared against a database of restriction maps from known organisms in order to identify the closest match to a restriction fragment pattern occurring in the database. This process can be repeated iteratively until sufficient matches are obtained to identify an organism at a predetermined confidence level. According to methods of the invention, nucleic acid from a sample are prepared and imaged as described herein. A restriction map is prepared and the restriction pattern is correlated with a database of restriction patterns for known organisms. In a preferred embodiment, organisms are identified from a sample containing a mixture of organisms. Use of methods of the invention allows the detection of multiple microorganisms from the same sample, either serially or simultaneously.

In use, the invention can be applied to identify or classify a microorganism making up a contaminant in an environmental sample. For example, methods of the invention are useful to identify a potential biological hazard in a sample of air, water, soil, clothing, luggage, saliva, urine, blood, sputum, food, drink, and others. In a preferred embodiment, methods of the invention are used to detect and identify an organism in a sample obtained from an unknown source. In essence, methods of the invention can be used to detect biohazards in any environmental or industrial setting.

Further aspects and features of the invention will be apparent upon inspection of the following detailed description thereof.

All patents, patent applications, and references cited herein are incorporated in their entireties by reference.

### BRIEF DESCRIPTION OF THE DRAWINGS

FIG. 1 is a diagram showing restriction maps of six isolates of E. coli.

FIG. 2 is a diagram showing restriction maps of six isolates of E. coli clustered into three groups: O157 (that includes O157:H7 and 536), CFT (that includes CFT073 and 1381), and K12 (that includes K12 and 718).

FIG. 3 is a diagram showing common motifs among restriction maps of six isolates of E. coli.

FIG. 4 is a diagram showing restriction maps of six isolates of E. coli, with the boxes indicating regions common to E. coli.

FIG. 5 is a diagram showing restriction maps of six isolates of E. coli, with the boxes indicating regions that are unique to a particular strain, namely O157, CFT, or K12.

FIG. 6 is a diagram showing restriction maps of six isolates of E. coli, with the boxes indicating regions unique to each isolate.

FIG. 7 is a tree diagram, showing possible levels of identifying E. coli.

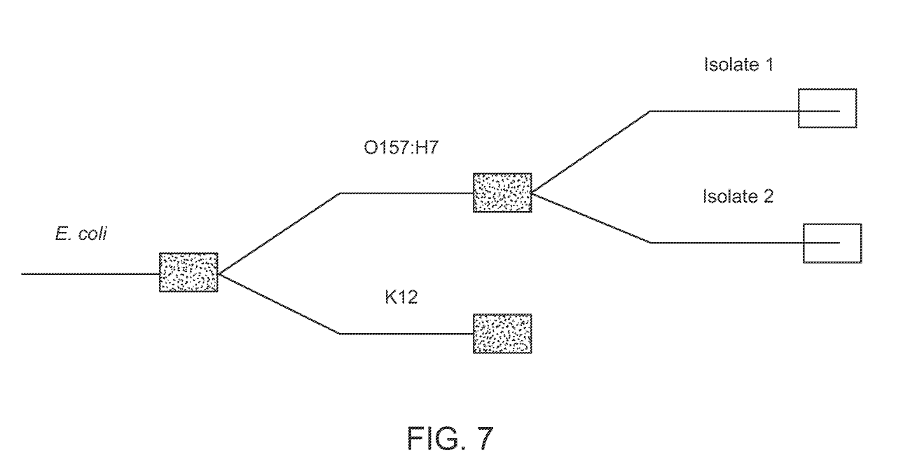

FIG. 8 is a diagram showing restriction maps of a sample (middle map) and related restriction maps from a database.

FIG. 9 is a schematic diagram showing patterns of markers in various vancomycin-resistant bacterial strains in which dark triangles represent deletions, lighter triangles represent insertions, semicircular arrows are inversions, and double arrows are tandem repeats.

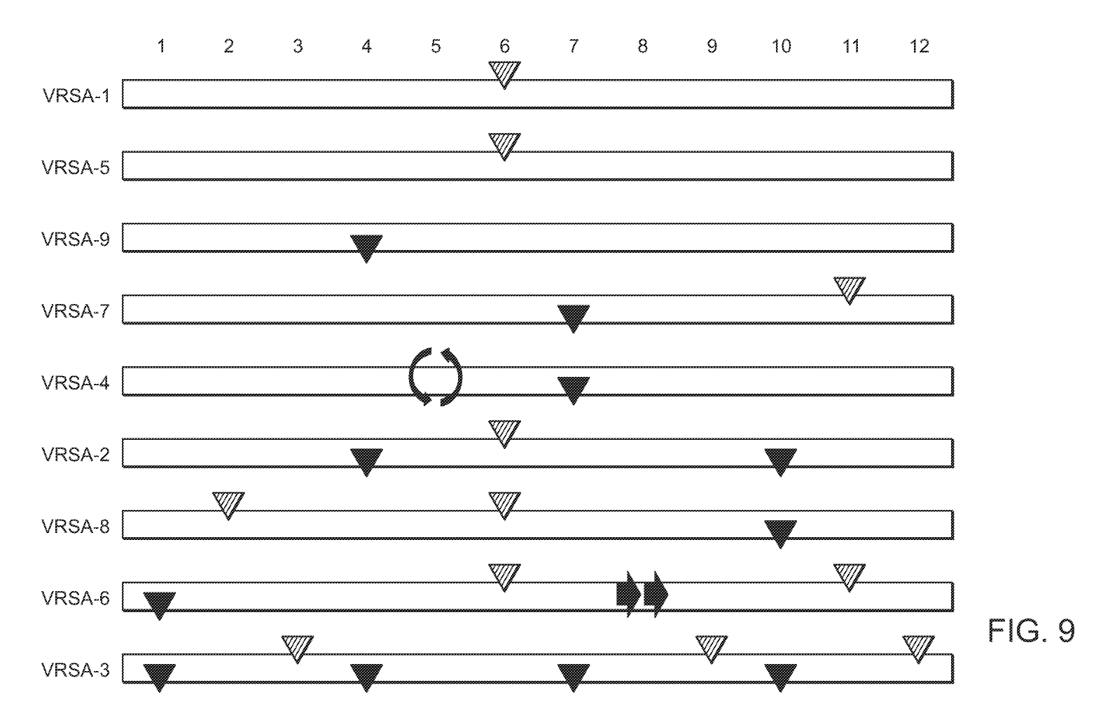

FIG. 10 is a comparison of a methicillin-resistant bacterium and three different strains of vancomycin-resistant bacteria, showing restriction fragment patterns from optical maps according to the invention.

FIG. 11 shows pattern matching between two methicillin-resistant bacteria and a vancomycin-resistant bacterium from optical maps prepared according to the invention.

FIG. 12 is a schematic diagram showing patterns of markers in various methicillin-resistant Staphylococcus aureaus strains.

### DETAILED DESCRIPTION

The present invention provides methods of identifying and/or classifying mircoorganisms. Preferred methods include obtaining a restriction map of a nucleic acid, e.g., DNA, from each organism and correlating the restriction map of each nucleic acid with a restriction map database, thereby identifying and/or comparing organisms obtained from a sample. With use of a detailed restriction map database that contains motifs common to various groups and sub-groups, organisms can be identified and classified not just at a genus and species level, but also at a sub-species (strain), a sub-strain, and/or an isolate level. For example, bacteria can be identified and classified at a genus level, e.g., Escherichia genus, species level, e.g., E. coli species, a strain level, e.g., O157, CFT, and K12 strains of E. coli, and isolates, e.g., O157:H7 isolate of E. coli (as described in Experiment 3B below). The featured methods offer a fast, accurate, and detailed information for identifying organisms. These methods can be used in a variety of clinical settings, e.g., for identification of an organism in a subject, e.g., a human or an animal subject.

This disclosure also features methods of diagnosing a disease or disorder in a subject by, inter alia, identifying each organism in a sample, including a heterogeneous sample, via correlating the restriction map of a nucleic acid from each organism with a restriction map database, and correlating the identity of each organism in the sample with the disease or disorder. These methods can be used in a clinical setting, e.g., human or veterinary setting.

Methods of the invention are also useful for identifying and/or detecting organisms in food or in an environmental setting. For example, methods of the invention can be used to assess an environmental threat in drinking water, air, soil, and other environmental sources. Methods of the invention are also useful to identify organisms in food and to determine a common source of food poisoning in multiple samples that are separated in time or geographically, as well as samples that are from the same or similar batches.

In a particularly-preferred embodiment, methods of the invention comprise identifying restriction patterns based upon optical mapping and using those patterns to determine characteristics of the organism being analyzed. For example, a microorganism is compared to a database of known patterns in order to determine properties that allow identification of the organism, characteristics of the organism, classification of the organism, and other features that aid in, for example, disease diagnosis and treatment.

### Restriction Mapping

Methods featured herein utilize restriction mapping during both generation of the database and processing of an organism to be identified. One type of restriction mapping that is used is optical mapping. Optical mapping is a echnique for production of ordered restriction maps from a single DNA molecule (Samad et al., Genome Res. 5:1-4, 1995). During this method, fluorescently labeled DNA molecules are elongated in a flow of agarose between a coverslip and a microscope slide (in the first-generation method) or fixed onto polylysine-treated glass surfaces (in a second-generation method). Id. The added endonuclease cuts the DNA at specific points, and the fragments are imaged. Id. Restriction maps can be constructed based on the number of fragments resulting from the digest. Id. Generally, the final map is an average of fragment sizes derived from similar molecules. Id. Thus, in one embodiment of the present methods, the restriction map of an organism to be identified is an average of a number of maps generated from the sample containing the organism.

Optical mapping and related methods are described in U.S. Pat. No. 5,405,519, U.S. Pat. No. 5,599,664, U.S. Pat. No. 6,150,089, U.S. Pat. No. 6,147,198, U.S. Pat. No. 5,720,928, U.S. Pat. No. 6,174,671, U.S. Pat. No. 6,294,136, U.S. Pat. No. 6,340,567, U.S. Pat. No. 6,448,012, U.S. Pat. No. 6,509,158, U.S. Pat. No. 6,610,256, and U.S. Pat. No. 6,713,263, each of which is incorporated by reference herein. Optical Maps are constructed as described in Reslewic et al., Appl Environ Microbiol. 2005 September; 71 (9):5511-22, incorporated by reference herein. Briefly, individual chromosomal fragments from test organisms are immobilized on derivatized glass by virtue of electrostatic interactions between the negatively-charged DNA and the positively-charged surface, digested with one or more restriction endonuclease, stained with an intercalating dye such as YOYO-1 (Invitrogen) and positioned onto an automated fluorescent microscope for image analysis. Since the chromosomal fragments are immobilized, the restriction fragments produced by digestion with the restriction endonuclease remain attached to the glass and can be visualized by fluorescence microscopy, after staining with the intercalating dye. The size of each restriction fragment in a chromosomal DNA molecule is measured using image analysis software and identical restriction fragment patterns in different molecules are used to assemble ordered restriction maps covering the entire chromosome.

### Restriction Map Database

The database(s) used with methods described herein are generated by optical mapping techniques discussed supra. The database(s) can contain information for a large number of isolates, e.g., about 200, about 300, about 400, about 500, about 600, about 700, about 800, about 900, about 1,000, about 1,500, about 2,000, about 3,000, about 5,000, about 10,000 or more isolates. In addition, the restriction maps of the database contain annotated information (a similarity cluster) regarding motifs common to genus, species, sub-species (strain), sub-strain, and/or isolates for various organisms. The large number of the isolates and the information regarding specific motifs allows for accurate and rapid identification of an organism.

The restriction maps of the database(s) can be generated by digesting (cutting) nucleic acids from various isolates with specific restriction endonuclease enzymes. Some maps can be a result of digestion with one endonuclease. Some maps can be a result of a digest with a combination of endonucleases, e.g., two, three, four, five, six, seven, eight, nine, ten or more endonucleases. The exemplary endonucleases that can be used to generate restriction maps for the database(s) and/or the organism to be identified include: BglII, NcoI, XbaI, and BamHI. Non-exhaustive examples of other endonucleases that can be used include: Alul, ClaI, DpnI, EcoRI, HindIII, KpnI, PstI, SacI, and SmaI. Yet other restriction endonucleases are known in the art.

Map alignments between different strains are generated with a dynamic programming algorithm which finds the optimal alignment of two restriction maps according to a scoring model that incorporates fragment sizing errors, false and missing cuts, and missing small fragments (See Myers et al., Bull Math Biol 54:599-618 (1992); Tang et al., J Appl Probab 38:335-356 (2001); and Waterman et al., Nucleic Acids Res 12:237-242). For a given alignment, the score is proportional to the log of the length of the alignment, penalized by the differences between the two maps, such that longer, better-matching alignments will have higher scores.

To generate similarity clusters, each map is aligned against every other map. From these alignments, a pair-wise alignment analysis is performed to determine “percent dissimilarity” between the members of the pair by taking the total length of the unmatched regions in both genomes divided by the total size of both genomes. These dissimilarity measurements are used as inputs into the agglomerative clustering method “Agnes” as implemented in the statistical package “R”. Briefly, this clustering method works by initially placing each entry in its own cluster, then iteratively joining the two nearest clusters, where the distance between two clusters is the smallest dissimilarity between a point in one cluster and a point in the other cluster.

### Organisms to be Identified

Various organisms, e.g., viruses, and various microorganisms, e.g., bacteria, protists, and fungi, can be identified with the methods featured herein. In one embodiment, the organism's genetic information is stored in the form of DNA. The genetic information can also be stored as RNA.

The sample containing the organism to be identified can be a human sample, e.g., a tissue sample, e.g., epithelial (e.g., skin), connective (e.g., blood and bone), muscle, and nervous tissue, or a secretion sample, e.g., saliva, urine, tears, and feces sample. The sample can also be a non-human sample, e.g., a horse, camel, llama, cow, sheep, goat, pig, dog, cat, weasel, rodent, bird, reptile, and insect sample. The sample can also be from a plant, water source, food, air, soil, plants, or other environmental or industrial sources.

### Identifying Organisms

The methods described herein, i.e., methods of identifying at least one organism, diagnosing a disease or disorder in a subject, determining antibiotic resistance of at least one organism, determining an antibiotic resistance profile of a bacterium, and determining a therapeutically effective antibiotic to administer to a subject, and treating a subject, include correlating the restriction map of a nucleic acid of each organism with a restriction map database. The methods involve comparing each of the raw single molecule maps from the unknown sample (or an average restriction map of the sample) against each of the entries in the database, and then combining match probabilities across different molecules to create an overall match probability.

In one embodiment of the methods, entire genome of the organism to be identified can be compared to the database. In another embodiment, several methods of extracting shared elements from the genome can be created to generate a reduced set of regions of the organism's genome that can still serve as a reference point for the matching algorithms.

As discussed above and in the Examples below, the restriction maps of the database can contain annotated information (a similarity cluster) regarding motifs common to genus, species, sub-species (strain), sub-strain, and/or isolates for various organisms. Such detailed information would allow identification of an organism at a sub-species level, which, in turn, would allow for a more accurate diagnosis and/or treatment of a subject carrying the organism.

In another embodiment, methods of the invention are used to identify genetic motifs that are indicative of an organism, strain, or condition. For example, methods of the invention are used to identify in an isolate at least one motif that confers antibiotic resistance. This allows appropriate choice of treatment without further cluster analysis.

### Applications

Methods described herein are used in a variety of settings, e.g., to identify an organism in a human or a non-human subject, in food, in environmental sources (e.g., food, water, air), and in industrial settings. The featured methods also include methods of diagnosing a disease or disorder in a subject, e.g., a human or a non-human subject, and treating the subject based on the diagnosis. The method includes: obtaining a sample comprising an organism from the subject; imaging a nucleic acid from the organism; obtaining a restriction map of said nucleic acid; identifying the organism by correlating the restriction map of said nucleic acid with a restriction map database; and correlating the identity of the organism with the disease or disorder.

As discussed above, various organisms can be identified by the methods discussed herein and therefore various diseases and disorders can be diagnosed by the present methods. The organism can be, e.g., a cause, a contributor, and/or a symptom of the disease or disorder. In one embodiment, more than one organism can be identified by the methods described herein, and a combination of the organisms present can lead to diagnosis. Skilled practitioners would be able to correlate the identity of an organism with a disease or disorder. For example, the following is a non-exhaustive list of some diseases and bacteria known to cause them: tetanus—Clostridium tetani; tuberculosis—Mycobacterium tuberculosis; meningitis—Neisseria meningitidis; botulism—Clostridium botulinum; bacterial dysentry—Shigella dysenteriae; lyme disease—Borrelia burgdorferi; gasteroenteritis—E. coli and/or Campylobacter spp.; food poisoning—Clostridium perfringens, Bacillus cereus, Salmonella enteriditis, and/or Staphylococcus aureus. These and other diseases and disorders can be diagnosed by the methods described herein.

Once a disease or disorder is diagnosed, a decision about treating the subject can be made, e.g., by a medical provider or a veterinarian. Treating the subject can involve administering a drug or a combination of drugs to ameliorate the disease or disorder to which the identified organism is contributing or of which the identified organism is a cause. Amelioration of the disease or disorder can include reduction in the symptoms of the disease or disorder. The drug administered to the subject can include any chemical substance that affects the processes of the mind or body, e.g., an antibody and/or a small molecule, The drug can be administered in the form of a composition, e.g., a composition comprising the drug and a pharmaceutically acceptable carrier. The composition can be in a form suitable for, e.g., intravenous, oral, topical, intramuscular, intradermal, subcutaneous, and anal administration. Suitable pharmaceutical carriers include, e.g., sterile saline, physiological buffer solutions and the like. The pharmaceutical compositions may be additionally formulated to control the release of the active ingredients or prolong their presence in the patient's system. Numerous suitable drug delivery systems are known for this purpose and include, e.g., hydrogels, hydroxmethylcellulose, microcapsules, liposomes, microemulsions, microspheres, and the like. Treating the subject can also include chemotherapy and radiation therapy.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

### EXAMPLES

### Example 1

### Microbial Identification Using Optical Mapping

Microbial identification (ID) generally has two phases. In the first, DNA from a number of organisms are mapped and compared against one another. From these comparisons, important phenotypes and taxonomy are linked with map features. In the second phase, single molecule restriction maps are compared against the database to find the best match.

Database Building and Annotation

Maps sufficient to represent a diversity of organisms, on the basis of which it will be possible to discriminate among various organisms, are generated. The greater the diversity in the organisms in the database, the more precise will be the ability to identify an unknown organism. Ideally, a database contains sequence maps of known organisms at the species and sub-species level for a sufficient variety of microorganisms so as to be useful in a medical or industrial context. However, the precise number of organisms that are mapped into any given database is determined at the convenience of the user based upon the desired use to which the database is to be put.

After sufficient number of microorganisms are mapped, a map similarity cluster is generated. First, trees of maps are generated. After the tree construction, various phenotypic and taxonomic data are overlaid, and regions of the maps that uniquely distinguish individual clades from the rest of the populations are identified. The goal is to find particular clades that correlate with phenotypes/taxonomies of interest, which will be driven in part through improvements to the clustering method.

Once the clusters and trees have been annotated, the annotation will be applied back down to the individual maps. Additionally, if needed, the database will be trimmed to include only key regions of discrimination, which may increase time performance.

Calling (Identifying) an Unknown

One embodiment of testing the unknowns involves comparing each of the raw single molecule maps from the unknown sample against each of the entries in the database, and then combining match probabilities across different molecules to create an overall match probability.

The discrimination among closely related organisms can be done by simply picking the most hits or the best match probability by comparing data obtained from the organism to data in the database. More precise comparisons can be done by having detailed annotations on each genome for what is a discriminating characteristic of that particular genome versus what is a common motif shared among several isolates of the same species. Thus, when match scores are aggregated, the level of categorization (rather than a single genome) will receive a probability. Therefore, extensive annotation of the genomes in terms of what is a defining characteristic and what is shared will be required.

In one embodiment of the method, entire genomes will be compared to all molecules. Because there will generally be much overlap of maps within a species, another embodiment can be used. In the second embodiment, several methods of extracting shared elements from the genome will be created to generate a reduced set of regions that can still serve as a reference point for the matching algorithms. The second embodiment will allow for streamlining the reference database to increase system performance.

### Example 2

### Using Multiple Enzymes for Microbial Identification

In one embodiment, the single molecule restriction maps from each of the enzymes will be compared against the database described in Example 1 independently, and a probable identification will be called from each enzyme independently. Then, the final match probabilities will be combined as independent experiments. This embodiment will provide some built-in redundancy and therefore accuracy for the process.

### INTRODUCTION

In general, optical mapping can be used within a specific range of average fragment sizes, and for any given enzyme there is considerable variation in the average fragment size across different genomes. For these reasons, it typically will not be optimal to select a single enzyme for identification of clinically-relevant microbes. Instead, a small set of enzymes will be chosen to optimize the probability that for every organism of interest, there will be at least one enzyme in the database suitable for mapping.

Selection Criteria

A first step in the selection of enzymes was the identification of the bacteria of interest. These bacteria were classified into two groups: (a) the most common clinically interesting organisms and (b) other bacteria involved in human health. The chosen set of enzymes must have at least one enzyme that cuts each of the common clinically interesting bacteria within the range of average fragment sizes suitable for detailed comparisons of closely related genomes (about 6-13 kb). Additionally, for the remaining organisms, each fragment must be within the functional range for optical mapping (about 4-20 kb). These limits were determined through mathematical modeling, directed experiments, and experience with customer orders. Finally, enzymes that have already been used for Optical Mapping were selected.

Suggested Set

Based upon the above criteria, the preliminary set consisted of the enzymes BglII, NcoI, and XbaI, which have been used for optical mapping. There are 28 additional sets that cover the key organisms with known enzymes, so in the event that this set is not adequate, there alternatives will be utilized (data not shown).

Final Steps

Because the analysis in Experiment 2 is focused on the sequenced genomes, prior to full database production, this set of enzymes will be tested against other clinically important genomes, which will be part of the first phase of the proof of principle study.

### Example 3

### Identification of E. coli

A. In one embodiment of a microbial identification method, nucleic acids of between about 500 and about 1,000 isolates will be optically mapped. Then, unique motifs will be identified across genus, species, strains, substrains, and isolates. To identify a sample, single nucleic acid molecules of the sample will be aligned against the motifs, and p-values assigned for each motif match. The p-values will be combined to find likelihood of motifs. The most specific motif will give the identification.

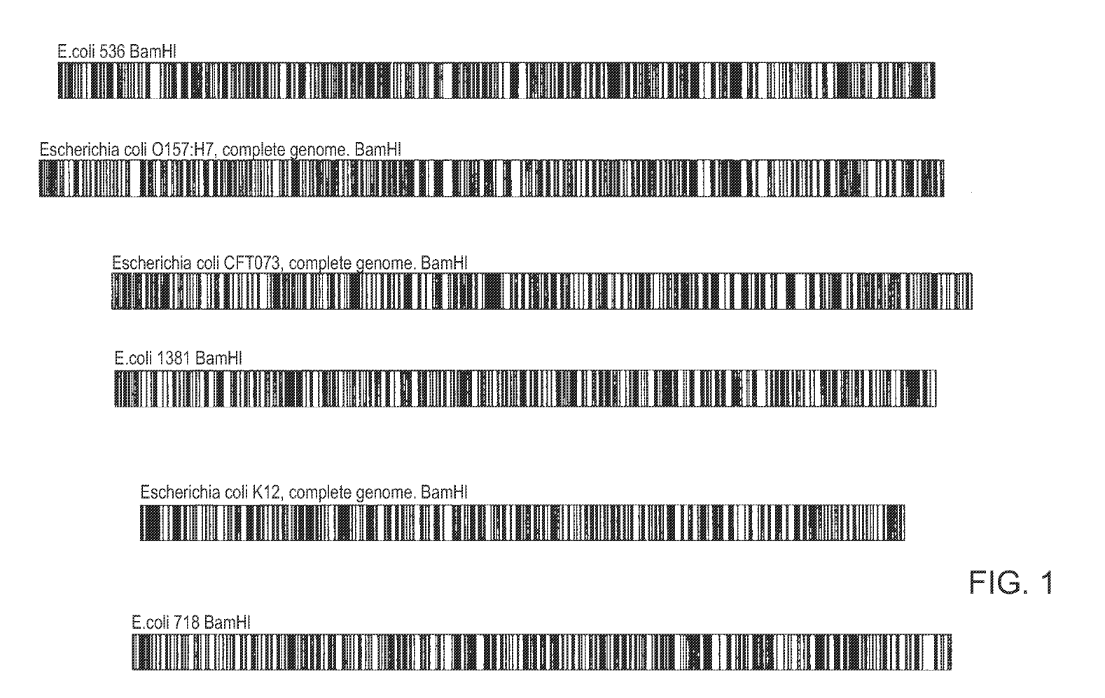

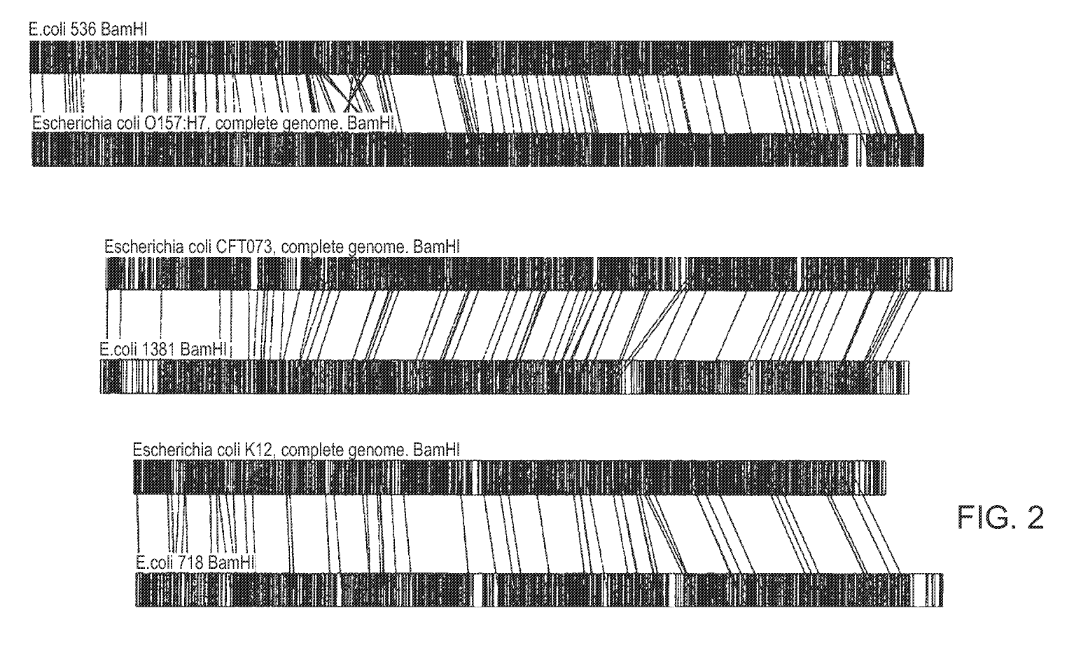

B. The following embodiment illustrates a method of identifying E. coli down to an isolate level. Restriction maps of six E. coli isolates were obtained by digesting nucleic acids of these isolates with BamHI restriction enzyme. FIG. 1 shows restriction maps of these six E. coli isolates: 536, O157:H7 (complete genome), CFT073 (complete genome), 1381, K12 (complete genome), and 718. As shown in FIG. 2, the isolates clustered into three sub-groups (strains): O157 (that includes O157:H7 and 536), CFT (that includes CFT073 and 1381), and K12 (that includes K12 and 718).

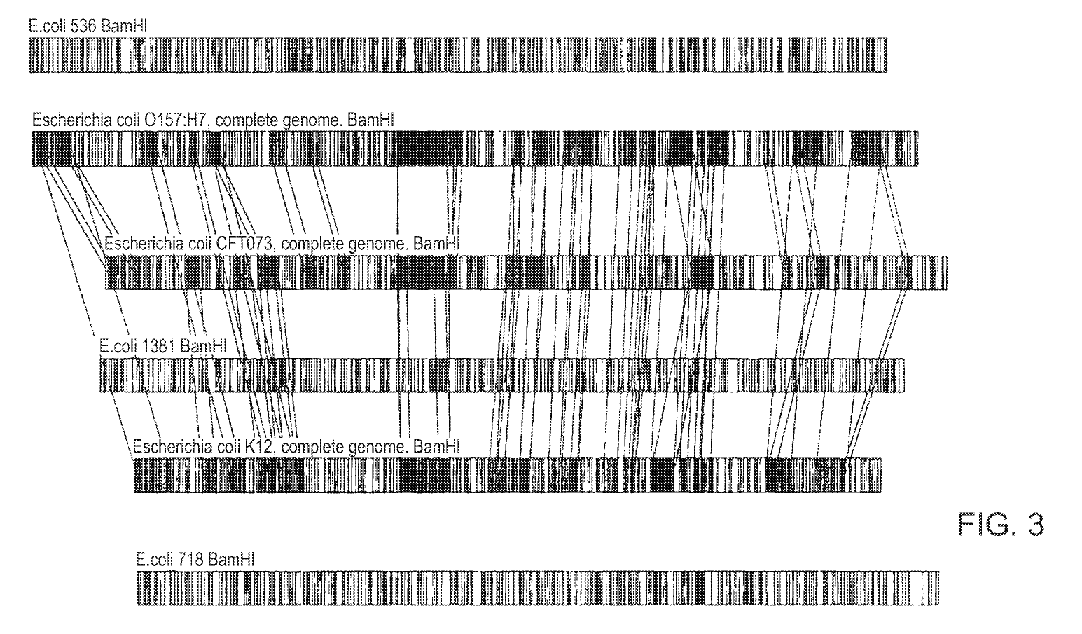

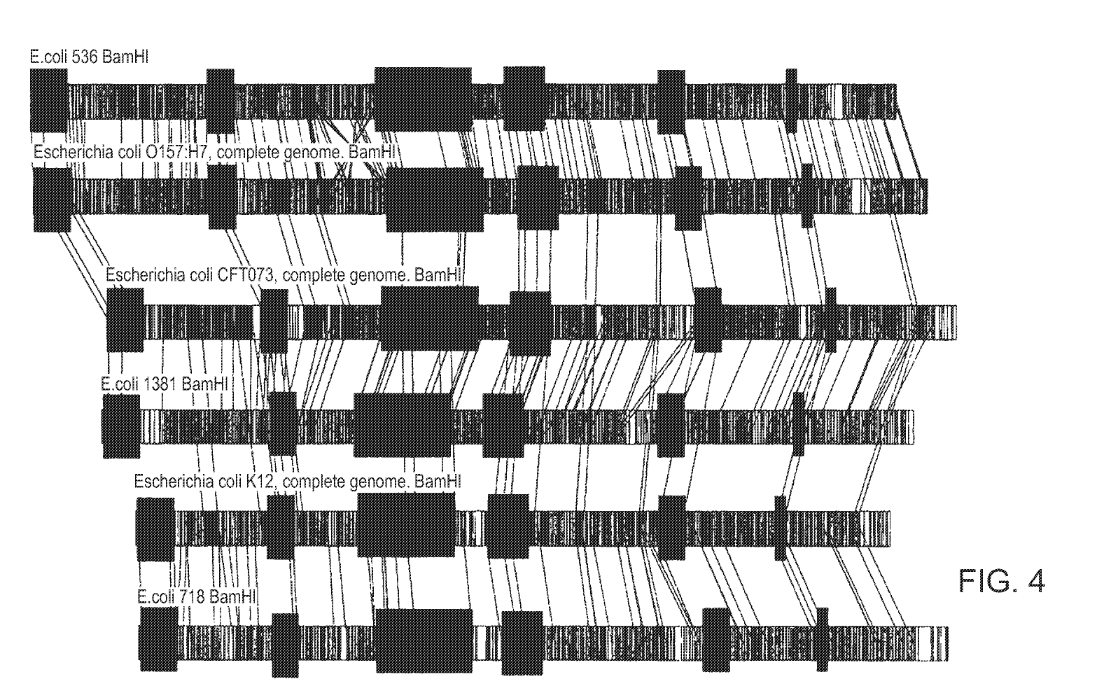

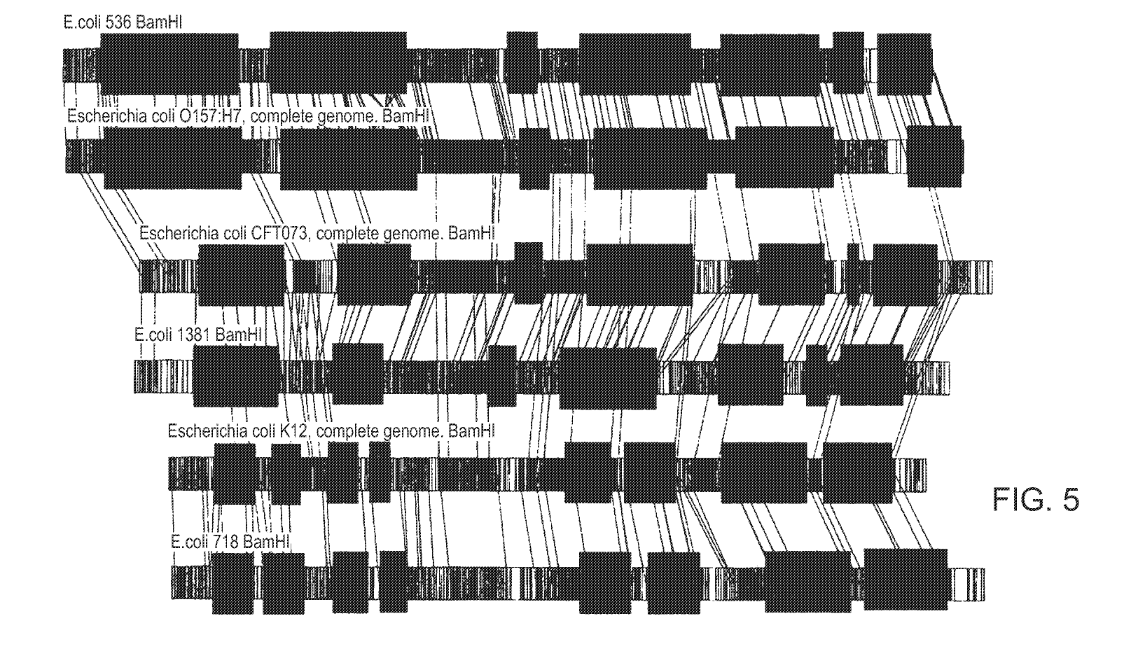

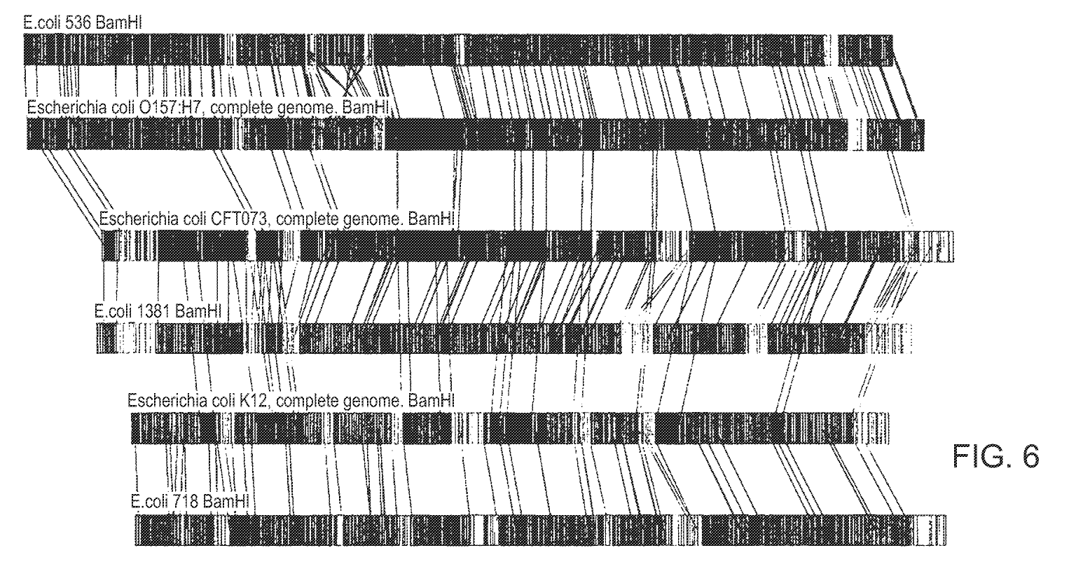

These restriction maps provided multi-level information regarding relation of these six isolates, e.g., showed motifs that are common to all of the three sub-groups (see, FIG. 3) and regions specific to E. coli (see, boxed areas in FIG. 4). The maps were also able to show regions unique to each strain (see, boxed areas in FIG. 5) and regions specific to each isolate (see boxed regions in FIG. 6).

This and similar information can be stored in a database and used to identify bacteria of interest. For example, a restriction map of an organism to be identified can be obtained by digesting the nucleic acid of the organism with BamHI. This restriction map can be compared with the maps in the database. If the map of the organism to be identified contains motifs specific to E. coli, to one of the sub-groups, to one of the strains, and/or to a specific isolate, the identity of the organism can be obtained by correlating the specific motifs. FIG. 6 shows a diagram to illustrate the possibilities of traversing variable lengths of a similarity tree.

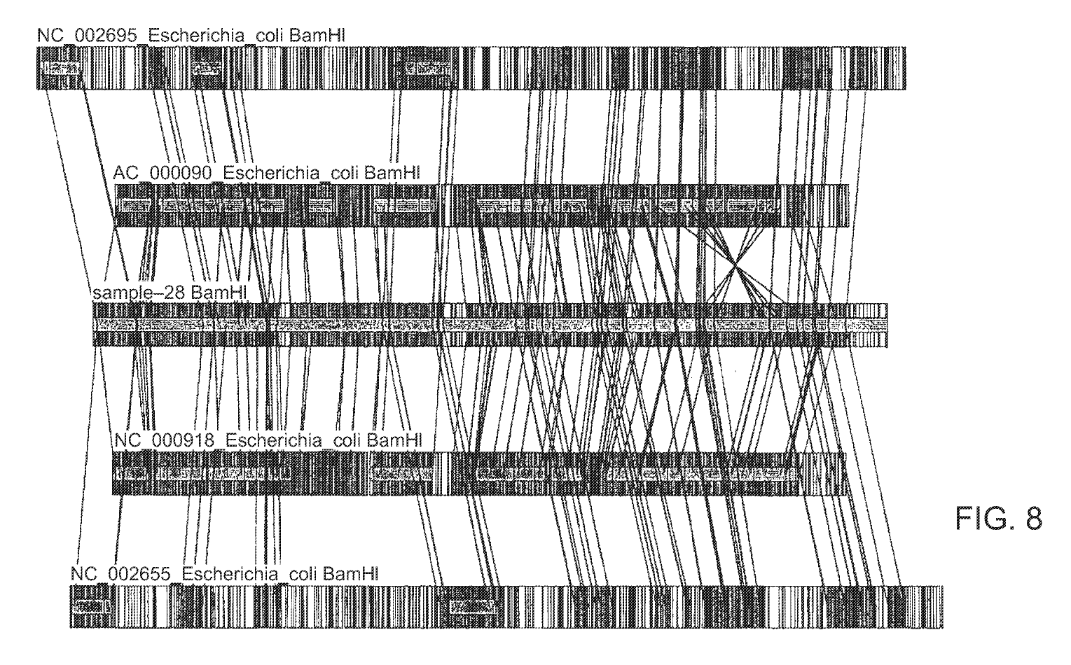

C. The following example illustrates identifying a sample as an E. coli bacterium. A sample (sample 28) was digested with BamHI and its restriction map obtained (see FIG. 8, middle restriction map). This sample was aligned against a database that contained various E. coli isolates. The sample was found to be similar to four E. coli isolates: NC 002695, AC 000091, NC 000913, and NC 002655. The sample was therefore identified as E. coli bacterium that is most closely related to the AC 000091 isolate.

### Example 4

### Identification of Bacteria from Clinical Samples

Rapid identification of bacteria is an important goal in clinical microbiology labs. Current testing procedures most often require pure culture, which significantly lengthens the time required for identification. In contrast, single molecule maps generated by Optical Mapping can theoretically provide more rapid identification, even when multiple organisms are present.

The example herein assessed the ability of Optical Mapping to identify unknown bacteria directly from clinical samples.

### Methods

Clinical samples were provided by Gundersen Lutheran Medical Foundation. The five samples for each of five clinical sample types (clinical colony, spiked blood bottles, spiked urine samples, clinical blood bottles, and clinical urine samples) were prepared and the identities blinded. Urine and blood culture bottle samples were processed by OpGen for isolation of bacterial cells. High molecular weight DNA for the samples were prepared directly from isolated bacterial cells using a modified Pulse-Field Gel Electrophoresis method as described in Birren et al. (Pulsed Field Gel Electrophoresis; A Practical Guide. San Diego: Academic Press, Inc. p. 25-74, 1993). Optical Chips for all DNA samples were prepared according to Reslewic et al. Microbial identification was performed by comparing collections of single molecule maps from each DNA sample to the identification database to determine the number of matches by using the algorithms described herein.

Results

DNA isolated from unknown samples from each of five sample type groups (clinical colony, spiked blood bottle, spiked urine sample, clinical blood bottle, and clinical urine sample) was analyzed by Optical Mapping using the restriction enzyme(s) specified. Collections of single molecule maps for each blinded clinical sample were analyzed using the algorithms described herein. Match data were generated using a p-value maximum set to 0.001. The number of single molecule maps that matched the top reported bacterial species as well as the next reported bacterial species from the ID are listed in Table 1 below. The final bacterial species identifications by Optical Mapping for each unknown sample along with the identifications made by Gundersen Luthern Medical Foundation microbiology laboratory are also represented.

TABLE 1
Clinical identification data

| | | | | Matches | | | | |
|---|---|---|---|---|---|---|---|---|
| | | | | to Top | Matches to | | | |
| | Unknown | | Top Reported | Reported | Next Reported | ID by Optical | | |
| Sample Type Group | Sample | Enzyme(s) | Species | Species | Species | Mapping | ID by GLMF | Results |
| Clinical Colony | UTI 1 | NcoI/Bg/II/XBaI | None | — | — | Not in DB | S. marcescens | Not in DB |
| Clinical Colony | UTI 2 | NcoI | E. coli | 55 | 0 | E. coli | E. coli | Correct |
| Clinical Colony | UTI 3 | Bg/II | E. coli | 51 | 1 | E. coli | E. coli | Correct |
| Clinical Colony | UTI 4 | NcoI | P. aenuginosa | 17 | 0 | P. aenuginosa | P. aenuginosa | Correct |
| Clinical Colony | UTI 5 | Bg/II | K. pneumoniae | 78 | 1 | K. pneumoniae | K. pneumoniae | Correct |
| Spiked Blood Bottle | SB 1 | NcoI | S. aureus | 64 | 0 | S. aureus | S. aureus | Correct |
| Spiked Blood Bottle | SB 2 | NcoI | E. Faecium | 86 | 1 | E. Faecium | E. Faecium | Correct |
| Spiked Blood Bottle | SB 3 | NcoI | S. pyogenes | 38 | 1 | S. pyogenes | S. pyogenes | Correct |
| Spiked Blood Bottle | SB 4 | Bg/II | P. auruginosa | 251 | 1 | P. auruginosa | P. auruginosa | Correct |
| Spiked Blood Bottle | SB 5 | NcoI | S. agalactiae | 122 | 2 | S. agalactiae | S. agalactiae | Correct |
| Spiked Urine Bottle | SU 1 | NcoI | E. coli | 186 | 2 | E. coli | E. coli | Correct |
| Spiked Urine Bottle | SU 2 | NcoI | P. mirabillis | 53 | 1 | P. mirabillis | P. mirabillis | Correct |
| Spiked Urine Bottle | SU 3 | NcoI | S. saprophyticus | 23 | 1 | S. saprophyticus | S. saprophyticus | Correct |
| Spiked Urine Bottle | SU 4 | Bg/II | K. pneumoniae | 66 | 1 | K. pneumoniae | K. pneumoniae | Correct |
| Spiked Urine Bottle | SU 5 | Bg/II | P. auruginosa | 71 | 1 | P. auruginosa | P. auruginosa | Correct |
| Clin. Blood Bottle | CB A | NcoI | S. epidermidis | 89 | 1 | S. epidermidis | S. epidermidis | Correct |
| Clin. Blood Bottle | CB B | NcoI | S. agalactiae | 19 | 0 | S. agalactiae | S. agalactiae | Correct |
| Clin. Blood Bottle | CB 3 | NcoI | E. coli | 22 | 1 | E. coli | E. coli | Correct |
| Clin. Blood Bottle | CB 4 | NcoI | K. pneumoniae | 15 | 2 | K. pneumoniae | K. pneumoniae | Correct |
| Clin. Blood Bottle | CB 6 | NcoI | E. coli | 100 | 1 | E. coli | E. coli | Correct |
| Clin. Urine Sample | CU 1 | NcoI | S. aureus | 200 | 1 | S. aureus | S. aureus | Correct |
| Clin. Urine Sample | CU 2 | NcoI | E. Faecalis | 69 | 1 | E. Faecalis | E. Faecalis | Correct |
| Clin. Urine Sample | CU 3 | NcoI | E. coli | 38 | 1 | E. coli | E. coli | Correct |
| Clin. Urine Sample | CU 4 | NcoI/Bg/II/XBaI | None | — | — | Not in DB | C. freundii | Not in DB |
| Clin. Urine Sample | CU 5 | Bg/II | *K. pneumoniae | 1 | 1 | K. pneumoniae | K. pneumoniae | Correct |

Comparison of the columns entitled “ID by Optical Mapping” and “ID by GLMF” show that Optical Mapping made the same identification as Gundersen Luthern Medical Foundation in all but two cases. The results column shows Optical Mapping called the correct bacterial species for the unknown samplein all but two cases. An * symbol represents an unknown sample where the Optical Mapping assembly was used instead of the microbial identification to make an identification.

Data herein showed that of the 23 clinical samples that contained a representative species in the identification database, 100% identified to the same species as was identified by classical microbiology techniques at the Gundersen Lutheran Medical Foundation laboratory (Table 3). Furthermore, UTI 1 and CU 4 were correctly identified as not being in the identification database (Table 3).

Thus data herein demonstrated the ability of Optical Mapping to provide identification of clinically relevant bacteria directly from clinical samples. In addition, the results provided strong evidence that Optical Mapping could be used to significantly reduce the time necessary to identify bacteria in a clinical laboratory.

### Example 5

### Identification of Bacteria from Heterogeneous Samples

An important goal of clinical microbiology laboratories is the rapid identification of bacteria from clinical samples. However, lengthy culturing steps to obtain enough of a pure culture to allow for identification will slow the time to a result. In contrast, Optical Mapping can potentially provide identifications directly from clinical samples that may contain more than a single organism thereby decreasing the time to a result.

The example herein assessed the ability of Optical Mapping to identify unknown bacteria in complex mixtures.

Methods

Bacterial mixes were provided by Gundersen Lutheran Medical Foundation. Bacterial species for the mixtures were normalized to 1×10⁹ CFU/ml and mixed in combinations and amounts to yield eight groups with varying constituents and ratios as shown in Table 2. The eight bacterial mixtures (1-8) were prepared with two to four bacterial species to allow for a specific ratio of each bacterium as measured by colony forming units. The percentage of each bacterium within each group is listed in Table 2.

TABLE 2
Mixed culture constituents and ratios

| Group | Bacterial Species | Percent |
|---|---|---|
| 1 | Escherichia coli O157:h7 ATCC 35150 | 50 |
| | Pseudomonas aeruginosa ATCC 9027 | 50 |
| 2 | Esherichia coli O157:h7 ATCC 35150 | 90 |
| | Pseudomonas aeruginosa ATCC 9027 | 10 |
| 3 | Staphylococcus aureus ATCC 25923 | 50 |
| | Escherichia coli O157:h7 ATCC 35150 | 50 |
| 4 | Staphylococcus aureus ATCC 25923 | 90 |
| | Escherichia coli O157:h7 ATCC 35150 | 10 |
| 5 | Staphylococcus aureus ATCC 25923 | 33 |
| | Escherichia coli O157:h7 ATCC 35150 | 33 |
| | Pseudomonas aeruginosa ATCC 9027 | 33 |
| 6 | Staphylococcus aureus ATCC 25923 | 60 |
| | Escherichia coli O157:h7 ATCC 35150 | 30 |
| | Pseudomonas aeruginosa ATCC 9027 | 10 |
| 7 | Enterococcus faecalis ATCC 19433 | 25 |
| | Staphylococcus aureus ATCC 25923 | 25 |
| | Escherichia coli O157:h7 ATCC 35150 | 25 |
| | Pseudomonas aeruginosa ATCC 9027 | 25 |
| 8 | Enterococcus faecalis ATCC 19433 | 50 |
| | Staphylococcus aureus ATCC 25923 | 20 |
| | Escherichia coli O157:h7 ATCC 35150 | 20 |
| | Pseudomonas aeruginosa ATCC 9027 | 10 |

High molecular weight DNA for the samples was prepared directly from isolated bacterial cells using a modified Pulse-Field Gel Electrophoresis method as described in Birren et al. Optical Chips for DNA samples were prepared according to Reslewic et al. Microbial identification was performed by comparing collections of single molecule maps from each DNA sample to the identification database to determine the number of matches by using the algorithms described herein.

Results

DNA isolated from eight unknown bacterial mixtures (A, B, C, D, E, F, G, and H) was analyzed by Optical Mapping using the enzyme(s) specified (NcoI, BglII). Collections of single molecule maps for each unknown mixture (Table 2) were analyzed using the algorithms described herein. The algorithms identified matches to the identification database (Table 3).

TABLE 3
Microbial mixture identification data

| | | | | | | Max | | |
|---|---|---|---|---|---|---|---|---|
| | | | | | | Matches to | | |
| Unknown | | S. aureus | E. coli | E. faecalis | P. aeruginosa | Untested | OpGen 1^{st} | OpGen 2^{nd} |
| Mix | Enzyme | Matches | Matches | Matches | Matches | Species | Choice | Choice |
| A | NcoI | 1330* | 204* | 1 | 0 | 3 | 4⁺ | 3 |
| | Bg/II | 1* | 78* | 0 | 1 | 2 | | |
| B | NcoI | 0 | 594* | 0 | 0* | 2 | 2⁺ | 1 |
| | Bg/II | 0 | 912* | 0 | 32* | 3 | | |
| C | NcoI | 376* | 451* | 0 | 0* | 3 | 6⁺ | 5 |
| | Bg/II | 29* | 924* | 0 | 127* | 3 | | |
| D | NcoI | 425* | 656* | 90* | 0* | 4 | 8 | 7⁺ |
| | Bg/II | 5* | 198* | 0* | 49* | 3 | | |
| E | NcoI | 536* | 1115* | 170* | 0* | 2 | 7 | 8⁺ |
| | Bg/II | 0* | 280* | 0* | 80* | 3 | | |
| F | NcoI | 301* | 518* | 0 | 0* | 3 | 5⁺ | 6 |
| | Bg/II | 2* | 245* | 0 | 150* | 3 | | |
| G | NcoI | 235* | 923* | 0 | 0 | 2 | 3⁺ | 4 |
| | Bg/II | 3* | 413* | 0 | 3 | 4 | | |
| H | NcoI | 0 | 285* | 0 | 0* | 2 | 1⁺ | 2 |
| | Bg/II | 0 | 647* | 0 | 777* | 2 | | |

The match data was generated using a p-value maximum set to 0.01. Data were from representative Optical Chips. The number of matches represented how many single molecule maps matched the database to a specific species.
A * marked set indicates a match to a test species at a level of 8-fold or higher above background (i.e. max hit to untested species).
The ⁺ indicates where a correct group identification was made.

Data indicated that the bacterial constituents of the complex mixtures were identified correctly in 8 of 8 groups. Furthermore, the percentage of contributing bacterial species was identified correctly for 6 of the 8 groups.

Thus data herein demonstrated the ability of Optical Mapping to provide identification of clinically relevant bacteria in complex mixtures. In addition, the results provided strong evidence that Optical Mapping could be used to significantly reduce the time necessary to identify bacteria in a clinical laboratory.

### Example 6

### Comparison of Patterns Between Bacterial Strains

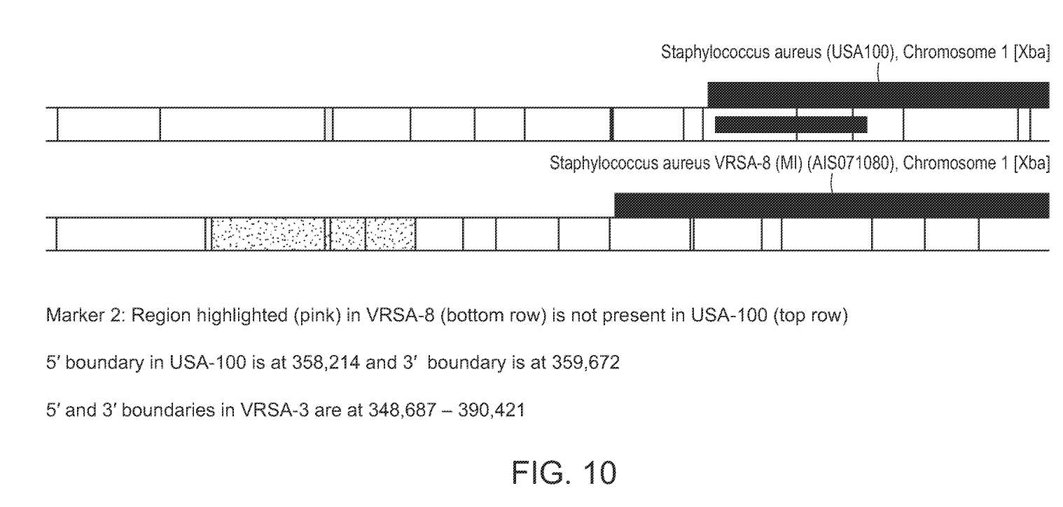

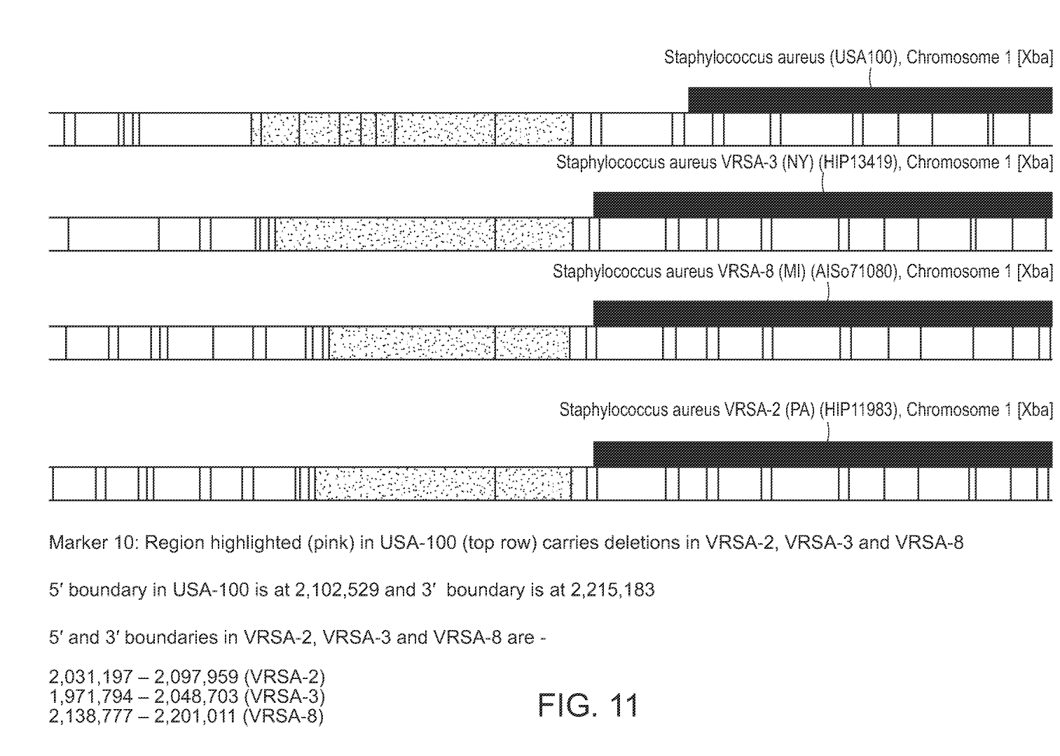

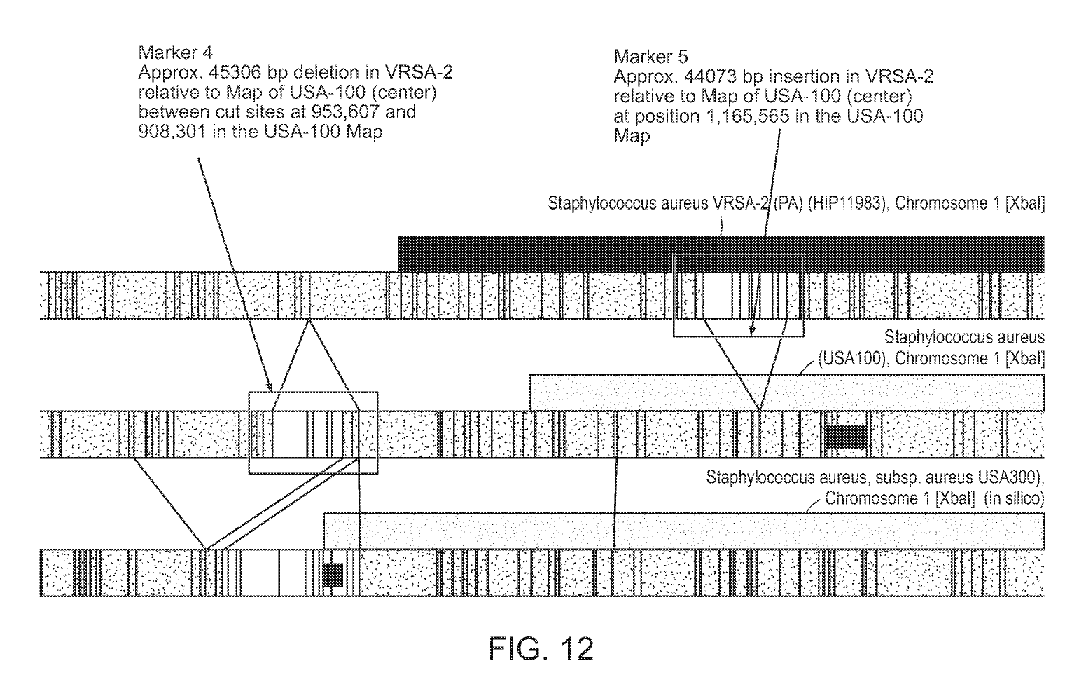

Several vancomycin-resistant Staphylococcus aureus (VRSA) and methicillin-resistant Staphylococcus aureas (MRSA) strains were obtained. The DNA was isolated and restriction digests were performed as provided above. An optical map was constructed using the methods described above for each strain and particular markers, or fragments, characteristic of the strains were identified. FIGS. 10-12 show the results for several of these comparisons. In FIG. 10, there clearly are unique restriction patterns (shown in pink) that differentiate the USA-100 MRSA and VRSA-8 strains. These patterns allow clear differentiation of those strains from each other. Referring to FIG. 11, the strains shown in that Figure enable classification of the three VRSA strains based upon an Xbal digest as VRSA-positive, but as different strains. However, the pattern is distinct from the MRSA strain shown immediately above, enabling easy distinction from the three VRSA strains. Finally, FIG. 12 shows how patterning according to the invention allows the indentification of two MRSA strains (USA 100 and USA 300) as MRSA and the VRSA-2 strain as a distinct strain. Indeed this is the case, as the MRSA and VRSA strains have different antibiotic resistance profiles that are indicated by the different restriction digest patterns revealed by optical mapping.

The embodiments of the disclosure may be carried out in ways other than those set forth herein without departing from the spirit and scope of the disclosure. The embodiments are, therefore, to be considered to be illustrative and not restrictive.

What is claimed is:

1. A method of categorizing an organism, the method comprising the steps of:
obtaining nucleic acid from an organism;
creating an optical map comprising a plurality of restriction fragments obtained from said nucleic acid;
comparing restriction fragment patterns in said map with optical map restriction fragment patterns obtained from at least one other organism; and
categorizing said organism based upon said comparing.

2. The method of claim 1, wherein the organism is a microorganism.

3. The method of claim 1, wherein the organism is a bacterium.

4. The method of claim 1, wherein the organism is a virus.

5. The method of claim 1, wherein the organism is a fungus.

6. The method of claim 1, wherein said nucleic acid sample comprises all genomic DNA of said organism.

7. The method of claim 1, wherein said nucleic acid sample comprises a transcriptome of said organism.

8. The method of claim 1, wherein said nucleic acid is deoxyribonucleic acid.

9. The method of claim 1, wherein said nucleic acid is ribonucleic acid.

10. The method of claim 1, wherein the organism is obtained from a human tissue or body fluid sample.

11. The method of claim 1, wherein the database comprises a restriction map similarity cluster.

12. The method of claim 1, wherein the database comprises a restriction map from at least one member of the clade of the organism.

13. The method of claim 1, wherein the database comprises a restriction map from at least one subspecies of the organism.

14. The method of claim 1, wherein the database comprises a restriction map from a genus, a species, a strain, a sub-strain, or an isolate of each organism.

15. The method of claim 1, wherein the database comprises a restriction map comprising motifs common to a genus, a species, a strain, a sub-strain, or an isolate of each organism.

16. A method of identifying a pathogen, the method comprising the steps of:
obtaining nucleic acid from a suspected pathogen;
creating an optical map of said nucleic acid;
comparing said optical map to at least one other optical map created from a known pathogen;
identifying said pathogen based upon similarities in patterns between said optical map and said other optical map.
